(19) United States
(12) Patent Application Publication
(10) Pub. No.: US 2025/0295328 A1
(43) Pub. Date: Sep. 25, 2025

(54) PATIENT ROOM GEO-FENCING USING LIDAR

(71) Applicant: Curbell Medical Products, Inc., Orchard Park, NY (US)

(72) Inventor: Thomas P. Kennedy, Lake View, NY (US)

(21) Appl. No.: 18/970,867

(22) Filed: Dec. 5, 2024

Related U.S. Application Data
(60) Provisional application No. 63/606,208, filed on Dec. 5, 2023.

(51) Int. Cl. A61B 5/11; G01S 7/481; G01S 17/86; G01S 17/89

(52) U.S. Cl. CPC A61B 5/1113; G01S 7/4816; G01S 7/4817; G01S 17/86; G01S 17/89

(57) ABSTRACT

In some embodiments, the present disclosure provides a method for establishing virtual positions of interest in a patient space. The method includes obtaining a baseline scan of the patient space using a LiDAR scanner. The baseline scan may be a 2D scan or a 3D scan. A signaling device is placed at a first location within the patient space. The method includes using the signaling device to signal a receiver to indicate the first location is a position of interest. A first scan of the patient device is obtained using the LiDAR scanner. A first virtual location of the signaling device within the patient space is determined based on the difference between the first scan and the baseline scan. A first position of interest is determined based on the first virtual location of the signaling device.

### CROSS-REFERENCE TO RELATED APPLICATIONS

This application claims priority to U.S. Provisional Application No. 63/606,208, filed on Dec. 5, 2023, now pending, the disclosure of which is incorporated herein by reference.

### FIELD OF THE DISCLOSURE

The present disclosure relates to techniques for fall management, and in particular, methods and systems for fall management in an inpatient setting.

### BACKGROUND OF THE DISCLOSURE

Many falls in hospital and rehabilitation settings result from unexpected or uncontrolled patient movement in a room—for example, unsupervised patient visits to a bathroom. However, it can be difficult to closely supervise a patient, especially to do so while maintaining the patient's privacy. For example, in some cases, video cameras may be used to monitor a patient's movement within the room.

There is a need for the ability to reduce the risk of falls by monitoring a patient's movement while also allowing the patient to maintain a level of privacy.

### BRIEF SUMMARY OF THE DISCLOSURE

In some embodiments, the present disclosure provides a method for establishing virtual positions of interest in a patient space. The method includes obtaining a baseline scan of the patient space using a LiDAR scanner. The baseline scan may be a 2D scan or a 3D scan. A signaling device is placed at a first location within the patient space. The method includes using the signaling device to signal a receiver to indicate the first location is a position of interest. For example, in some embodiments, the receiver may be an RF receiver, and signaling the receiver may be performed using an RF transmitter. In some embodiments, the receiver is a microphone, and signaling the receiver is performed using a sound (for example an audible tone, a word, a phrase, etc.) A first scan of the patient device is obtained using the LiDAR scanner. A first virtual location of the signaling device within the patient space is determined based on the difference between the first scan and the baseline scan. A first position of interest is determined based on the first virtual location of the signaling device.

The first position of interest may be an area (e.g., polygon, circle, ellipse, irregular shape, etc.) or a volume (e.g., polyhedron, sphere, ellipsoid, etc.), located based on the first location of the signaling device. In some embodiments, the first position of interest centered on the first location. In some embodiments, the first position of interest is offset from the first location. In some embodiments, the first location is the center (2D) or centroid (3D) of the signaling device. In some embodiments, the first position of interest is flagged as a keep out zone. In some embodiments, the first position of interest is flagged as a keep in zone.

In some embodiments, the method further includes labeling the first position of interest.

The method may further include moving the signaling device at a second location within the patient space; signaling the receiver to indicate the second location is a position of interest; obtaining a second scan of the patient space using the LiDAR scanner; identifying a second virtual location of the signaling device within the patient space based on a difference between the second scan and the baseline scan; and determining a second position of interest based on the second virtual location of the signaling device. In some embodiments, the first position of interest is merged with the second position of interest. In some embodiments, the method includes tracking a patient location relative to the first position of interest within the patient space. In some embodiments, the method includes providing an alert if the patient location is determined to be within the first position of interest and/or providing an alert if the patient location is determined to be outside of the first position of interest.

In some embodiments, the present disclosure may be embodied as a system for establishing virtual positions of interest in a patient space. The system includes a processor; a LiDAR scanner in electronic communication with the processor and configured to provide scans of the patient space; and a receiver in electronic communication with the processor. The processor may be configured to perform any of the methods disclosed herein. For example, the processor may be configured to: obtain a baseline scan of the patient space from the LiDAR scanner; obtain a first scan of the patient space when a trigger signal from a signaling device is received at the receiver; determine a first virtual location of the signaling device within the patient space based on the difference between the first scan and the baseline scan; and determine a first position of interest based on the first virtual location of the signaling device.

### DESCRIPTION OF THE DRAWINGS

For a fuller understanding of the nature and objects of the disclosure, reference should be made to the following detailed description taken in conjunction with the accompanying drawings.

FIG. 1A: A chart depicting a method according to an embodiment of the present disclosure.

FIG. 1B: A chart depicting a further embodiment of the method of FIG. 1A.

FIG. 2: A diagram of a system according to another embodiment of the present disclosure.

FIG. 3A: A diagram showing a merged position of interest formed by overlapping first position of interest and second position of interest and also a merged position of interest formed by discontinuous positions of interest (the merged position of interest is depicted by the heavy black outlines, the dashed oval is solely to illustrate the two discontinuous areas).

FIG. 3B: A diagram showing a consolidated position of interest made up of a perimeter formed using a plurality of positions of interest.

FIG. 4: A diagram showing a baseline map of office without people (2D LiDAR scan, scanner located approximately 1.5 meter above the floor).

FIG. 5: A diagram showing a person detected in the office of FIG. 4, the cluster of points indicating the person is highlighted by a min/max rectangle.

FIG. 6: A diagram showing the person in the office of FIGS. 4-5 and a series of square ‘keep out’ zones placed in a doorway (open door).

FIG. 7: A diagram showing the person in the office of FIGS. 4-6, where the person is shown near a ‘keep out’ zone, but not within the keep out zone.

FIG. 8: A diagram showing the person in the office of FIGS. 4-7 and depicting where the person has entered into ‘keep out’ zone and been detected (the bounding box around the person has changed in appearance to indicate the person has been detected in the keep out zone).

### DETAILED DESCRIPTION OF THE DISCLOSURE

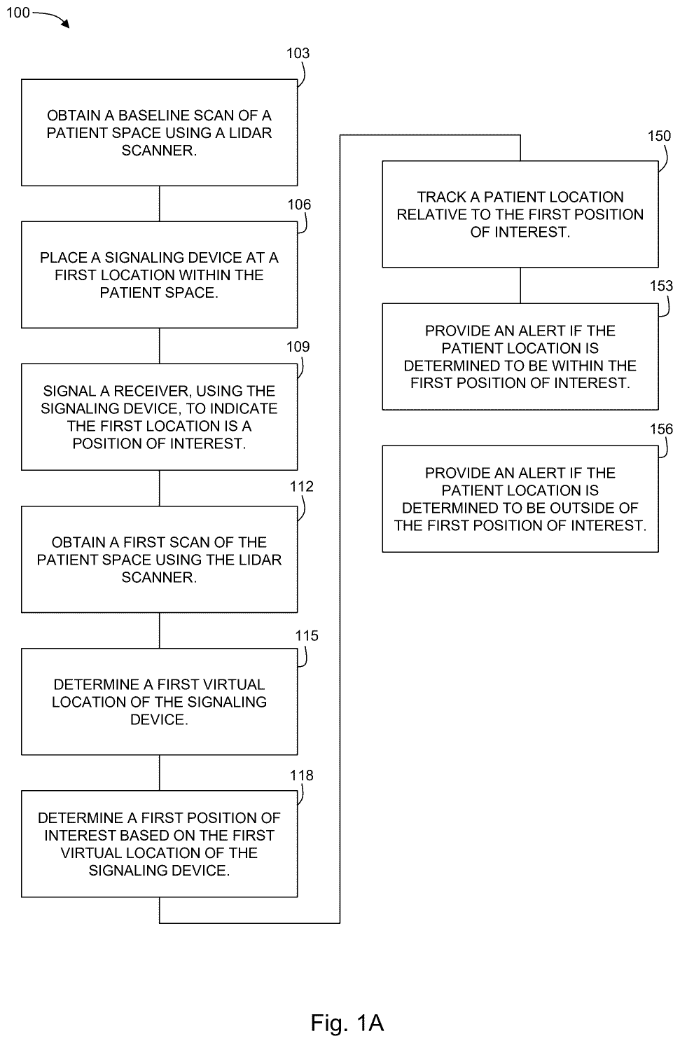

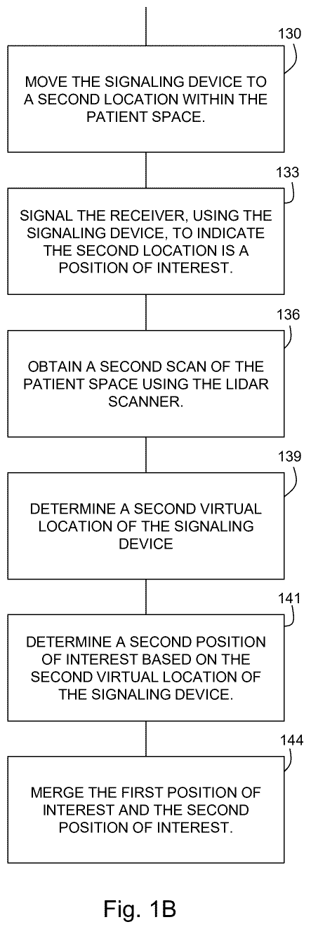

With reference to FIG. 1A, in an aspect, the present disclosure may be embodied as a method 100 for establishing virtual positions of interest in a patient space. The method 100 includes obtaining 103 a baseline scan of the patient space using a LiDAR scanner. For example, a LiDAR scanner may be mounted in a patient room in a position where most of the features (features of interest) in the room are in the Line of Site (LOS) of the LiDAR scanner. The baseline scan may be a two-dimensional (2D) scan or a three-dimensional (3D) scan. More than one LiDAR scanner may be used, and the resulting scans can be consolidated to provide a more comprehensive scan of the room (for example, where additional LiDAR scanners may be used to scan portions of the room hidden from the other LiDAR scanners). Non-limiting examples of features of interest may include sides or area around hospital bed(s), hand hygiene station(s) in a room, patient room doorway exit to the hospital, room doorway to bathroom in patient room.

A signaling device is placed 106 at a first location within the patient space. The signaling device is used to signal 109 a receiver to indicate the first location is a position of interest. For example, the receiver may be a radiofrequency (RF) receiver, and it may be signaled using an RF transmitter. In another example, the receiver is a microphone, and it may be signaled using a sound—for example, a pre-determined word or phrase.

A first scan of the patient space is obtained 112 using the LiDAR scanner. A first virtual location of the signaling device is determined 115. The first virtual location is determined based on the difference between the first scan and the baseline scan. In some embodiments, the first virtual location is at a center of the signaling device. In some embodiments, the first virtual location is at a centroid of the signaling device.

A first position of interest is determined 118 based on the first virtual location of the signaling device. The first position of interest may be a point in space, an area, or a volume. For example, the first position of interest may be predetermined to be a circular area centered at the first virtual location. In an example 3D scan, the first position of interest may be a volume (e.g., a cylindrical shape oriented to have a vertical longitudinal axis) extending the height of the scan and centered at the first virtual position. The first position of interest may have any shape, such as, for example, a polygon, circle, ellipse, polyhedron, sphere, ellipsoid, irregular shape, etc. In some embodiments, the first position of interest is centered at the first virtual location. In some embodiments, the first position of interest is offset from the first virtual location. For example, the first position of interest may be offset from the first virtual location by a predetermined distance and direction. For example, such an offset may be due to a shape of the transmitter device, use of the transmitter device, or other reason(s). In some embodiments, the first virtual location is determined to be the first position of interest.

The first position of interest may be labeled. For example, the first position of interest may be labeled for ease of user interaction—e.g., “bed”, “bathroom”, “kitchen”, etc.

The first position of interest may be flagged for use as a geo-fence. For example, the first position of interest may be flagged as a “keep-out” zone—i.e., where a signal is provided when a subject is located at/within the first position of interest. In another example, the first position of interest may be flagged as a “keep-in” zone—i.e., where a signal is provided when a subject is located outside of the first position of interest. These are intended to be non-limiting examples, and other uses for geo-fence information is within the scope of the present disclosure.

The method 100 may include tracking 150 a patient location relative to the first position of interest within the patient space. The method may include providing 153 an alert if the patient location is determined to be within the first position of interest. In some embodiments, the patient location is “within” the first position of interest when the patient location intersects with the first position of interest. For example, where the patient location is indicated by a point cloud and the first position of interest is indicated by a shape (e.g., rectangle, etc.) the patient location is within the first position of interest when a point of the point cloud intersects with the rectangle. In another example, where the patient location is indicated by a bounding shape (e.g., rectangle, etc.) and the first position of interest is indicated by another shape, the patient location is within the first position of interest when the two shapes intersect. The method may include providing 156 an alert if the patient location is determined to be outside of the first position of interest. Alerts may be in the form of an audible alarm, a visual alarm (e.g., a strobe light, etc.), a graphical display (e.g., on a display of a monitoring system, etc.), an alert sent to a healthcare provider (e.g., a text message, phone call, etc.), or other alerts or combinations of these and/or other alerts.

With reference to FIG. 1B, in some embodiments, the method 100 includes moving 130 the signaling device to a second location within the patient space. The receiver is signaled 133 to indicate the second location is a position of interest. A second scan of the patient space is obtained 136 using the LiDAR scanner. A second virtual location of the signaling device is identified 139 based on the difference between the second scan and the baseline scan. A second position of interest is determined 141 based on the second virtual location of the signaling device.

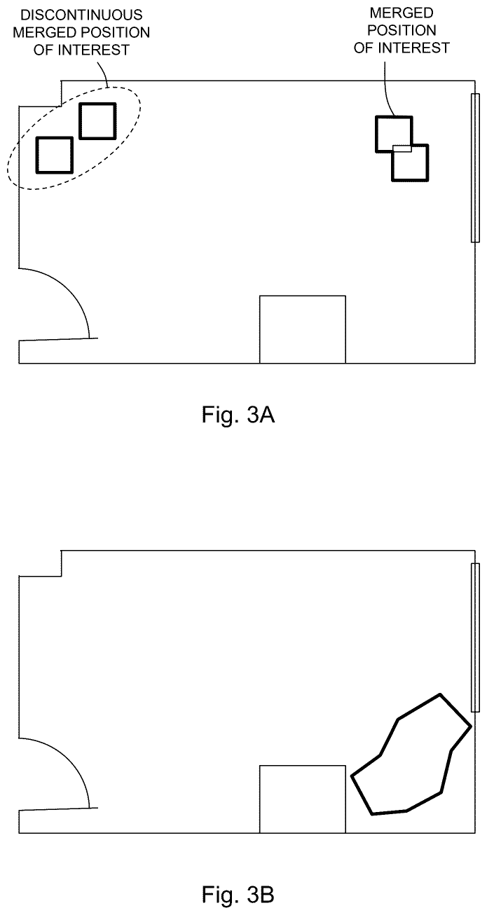

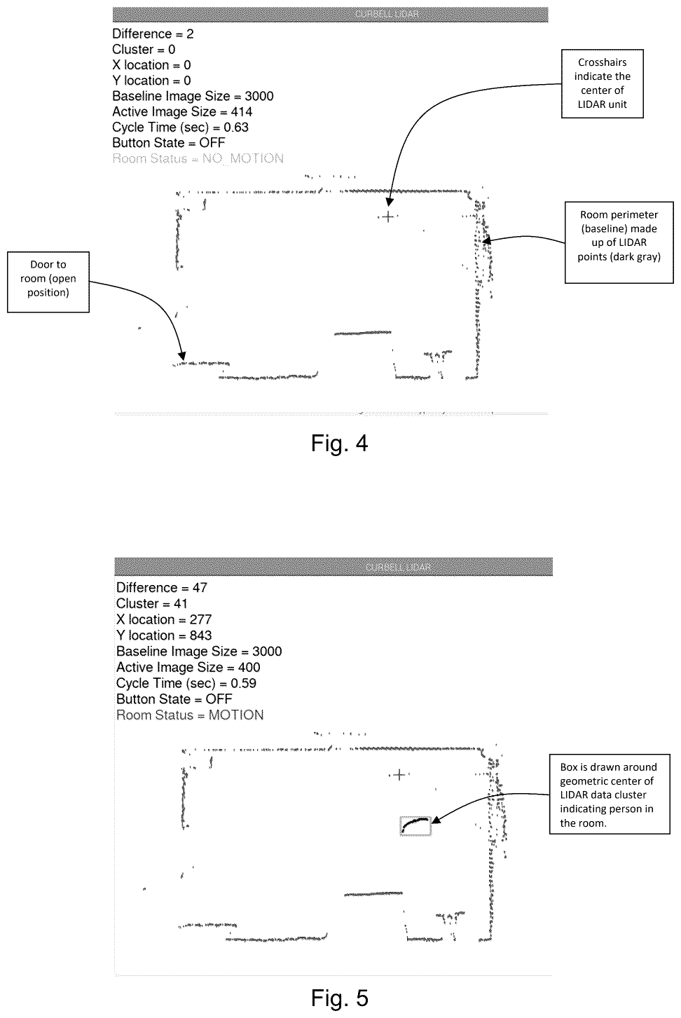

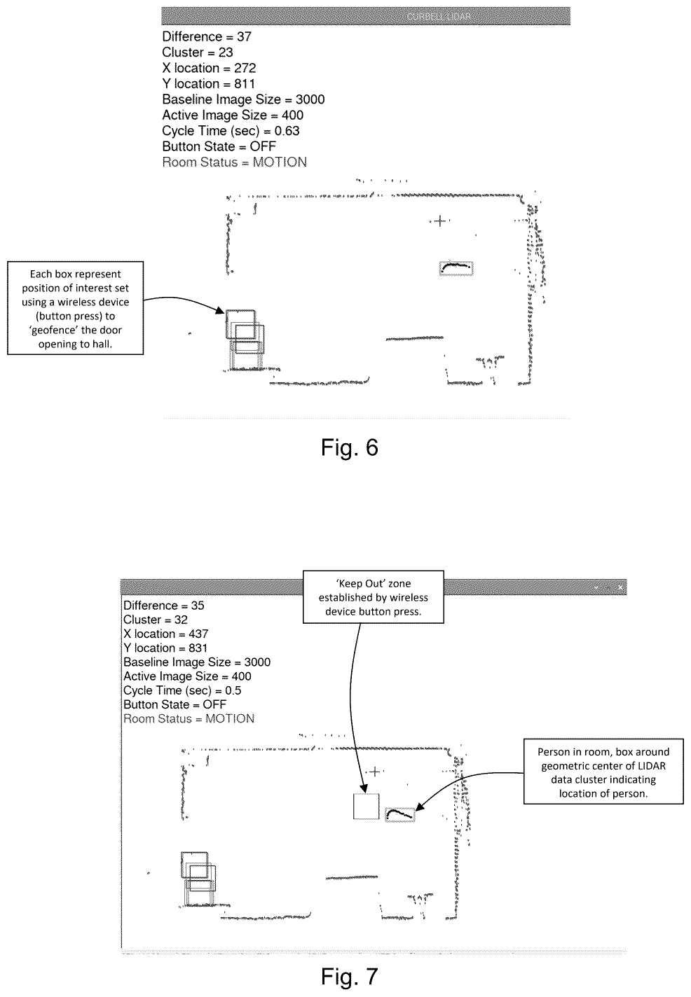

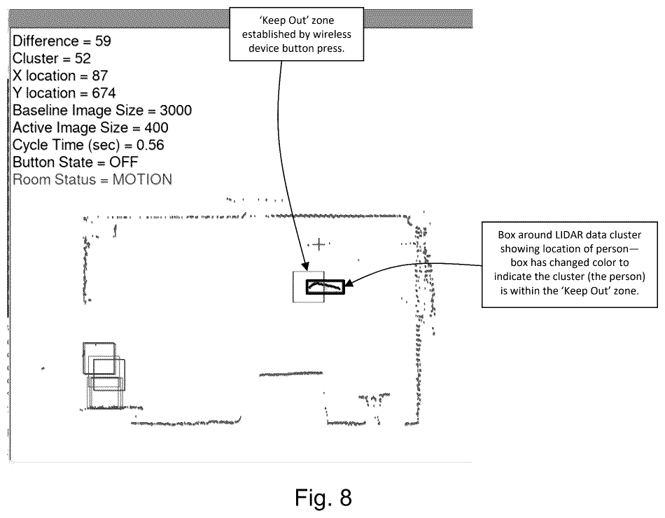

In some embodiments, the first position of interest and second position of interest are merged 144. For example, where the first and second positions of interest are areas, they may be merged into a larger area (e.g., FIG. 3A). In embodiments, where the first and second positions of interest do not overlap, the merged position of interest includes both of the separate areas (not continuous—e.g., FIG. 3A).

With reference to FIG. 3B, in some embodiments, a consolidated position of interest is determined based on the first position of interest and the second position of interest (and, potentially, additional positions of interest found in the same way as the first and second positions of interest). For example, the consolidated position of interest may be a geofence determined by a boundary drawn from each position of interest to the next, and ending by closing the boundary (i.e., back to the first position of interest). For example, each position of interest may be a point and the collection of points may be used to form a consolidated position of interest.

In some embodiments, the method includes moving the signaling device to one or more additional locations within the patient space. The receiver is signaled at each additional location to indicate the one or more additional locations are each a position of interest. Additional scans of the patient space are obtained using the LiDAR scanner, and one or more additional virtual locations of the signaling device are identified based on the difference between each additional scan and the baseline scan. One or more additional positions of interest are determined based on the corresponding one or more additional locations of the signaling device.

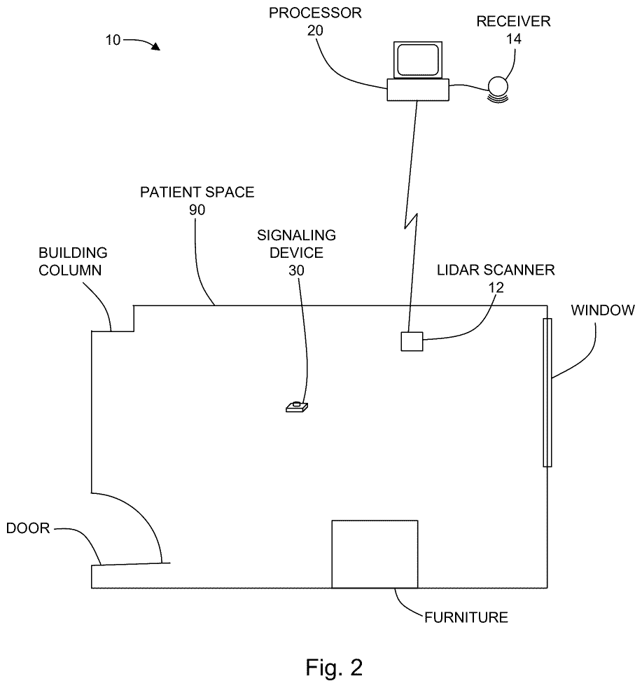

With reference to FIG. 2, in another aspect, the present disclosure may be embodied as a system 10 for establishing virtual positions of interest in a patient space. The system 10 includes a LiDAR scanner 12 configured to provide scans of a patient space 90. A processor 20 is in electronic communication with the LiDAR scanner 12. A receiver 14 is in electronic communication with the processor 20.

The processor 20 is configured (e.g., programmed) to perform any of the methods disclosed herein. For example, the processor 20 may be programmed to obtain a baseline scan of the patient space 90 from the LiDAR scanner 12. For example, the processor may cause the LiDAR scanner to obtain a scan (a baseline scan), and the LiDAR scanner may return a baseline scan to the processor. In some embodiments, the LiDAR scanner obtains more than one scan of the patient space and sends processed scan to the processor (e.g., averaged, denoised, etc.) In other embodiments, the processor may perform such pre-processing steps.

The receiver 14 may receive a trigger signal from a signaling device 30 within the patient space. The processor is programmed to obtain a first scan of the patient space when the receiver receives a trigger signal. For example, the processor may receive a signal from the receiver and then cause the LiDAR scanner to obtain a first scan, and the LiDAR scanner may return a first scan to the processor.

The processor is programmed to determine a first virtual location of the signaling device within the patient space based on a difference between the first scan and the baseline scan.

The processor is programmed to determine a first position of interest based on the first virtual location of the signaling device. The first position of interest may be a point in space, an area, or a volume. For example, the first position of interest may be predetermined to be a circular area centered at the first virtual location. In an example 3D scan, the first position of interest may be a volume (e.g., a cylindrical shape oriented to have a vertical longitudinal axis) extending the height of the scan and centered at the first virtual position. The first position of interest may have any shape, such as, for example, a polygon, circle, ellipse, polyhedron, sphere, ellipsoid, irregular shape, etc. In some embodiments, the first position of interest is centered at the first virtual location. In some embodiments, the first position of interest is offset from the first virtual location. For example, the first position of interest may be offset from the first virtual location by a predetermined distance and direction. For example, such an offset may be due to a shape of the transmitter device, use of the transmitter device, or other reason(s).

The first position of interest may be labeled. For example, the first position of interest may be labeled for ease of user interaction—e.g., “bed”, “bathroom”, “kitchen”, etc.

The first position of interest may be flagged for use as a geo-fence. For example, the first position of interest may be flagged as a “keep-out” zone—i.e., where a signal is provided when a subject is located at/within the first position of interest. In another example, the first position of interest may be flagged as a “keep-in” zone—i.e., where a signal is provided when a subject is located outside of the first position of interest. These are intended to be nonlimiting examples, and other uses for geo-fence information is within the scope of the present disclosure.

Software operation may include sequencing and timing, monitoring activities such as hand washing or trips to the bathroom.

### Further Discussion

The following provides further discussion of non-limiting embodiments and is intended solely to illustrate certain example details of the disclosure.

At initial system setup, the LiDAR device(s) scan the empty/static room environment, create and save a digital map or image of the room (baseline).

Once a digital map or image of the room is established, an individual enters and operates in the scanned space covered by the LiDAR device(s).

The system software determines the location of the individual in the monitored space by comparing current LiDAR scan data with the baseline map or image, recognizing the difference in LiDAR spatial data as the location of the individual within the space.

The individual is identified as a cluster of LiDAR spatial data points that are different from the baseline map or image; the size of the cluster needed to identify the individual can be used to differentiate the spatial data associated with the individual from ‘noise’ generated by imperfections in the baseline image or map.

The individual uses a digital switch capable of wireless communication with the LiDAR system to indicate a location of interest within the space by pressing the switch when physically located at the location of interest. The LiDAR system recognizes the switch state change and immediately determines the current position of the individual in the room as determined by the LiDAR spatial data. The system establishes the indicated position of the individual as a position of interest, adding a location marker of the position of interest to the digital map or image of the space.

The position of interest marker can be an area (ex: box or circle) or volume (cube or sphere/hemisphere), the dimensions of which can be variable.

The location of the position of interest with respect to the individual's body can be variable; a typical example may be the use of the average of the maximum and minimum Cartesian coordinates in 2 or 3 dimensions to determine the geometric center of the cluster of data points used to identify the individual in the space.

Once the location is established, a square area or cube volume, or circle or spherical shape can be generated references from the location. (ex: a rectangular area +/−50 mm in the cartesian x and y directions from the geometric center of the individual's data cluster)

Multiple positions of interest can be generated by the individual.

Positions of interest may be identified as ‘keep out’ and/or ‘keep in’ zones, as required by the application of the system. (e.g., the individual may use a two distinct button wireless switch, where pressing one button inserts a ‘keep out’ zone in the model, and the other switch inserts a ‘keep in’ zone in the model.

Larger zones of interest can be generated by placing multiple zones of interest in the digital map or image, or by varying the size or shape of the dimensional parameters for an individual zone.

The system may provide the ability to establish movement milestones within the room. As an example, a zone may be associated with a hand sanitizing station in a room and another zone associated with the patient bed in a room, and a third zone associated with the doorway of the patient room that opens into the hospital hallway. The system would monitor a caregiver or visitor entering a room from the hallway/doorway zone. If the caregiver or visitor move from the hallway/doorway zone without spending time in the hand sanitizing zone, an alarm or warning could be generated and/or broadcast, indicating that the caregiver/visitor must sanitize their hands before contacting the patient.

The system may provide the ability to establish acceptable movement time limits within a room. As an example, a zone may be associated with the patient bed and a zone may be associated with the bathroom door of a patient room. When the system may be able to determine that a patient has exited a bed and entered the bathroom, and start a timer. If the patient does not return to the bed in a predetermined amount of time, a warning or alarm may be generated to notify that the patient may need assistance.

The term processor is intended to be interpreted broadly. For example, in some embodiments, the processor includes one or more modules and/or components. Each module/component executed by the processor can be any combination of hardware-based module/component (e.g., graphics processing unit (GPU), a field-programmable gate array (FPGA), an application-specific integrated circuit (ASIC), a digital signal processor (DSP)), software-based module (e.g., a module of computer code stored in the memory and/or in the database, and/or executed at the processor), and/or a combination of hardware-and softwarebased modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general-purpose processor, a central processing unit (CPU), an accelerated processing unit (APU), a field-programmable gate array (FPGA), an application-specific integrated circuit (ASIC), a digital signal processor (DSP), graphics processing unit (GPU), microprocessor, controller, microcontroller, and/or the like.

Although the present disclosure has been described with respect to one or more particular embodiments, it will be understood that other embodiments of the present disclosure may be made without departing from the spirit and scope of the present disclosure.

1. A method for establishing virtual positions of interest in a patient space, comprising:
obtaining a baseline scan of the patient space using a LiDAR scanner;
placing a signaling device at a first location within the patient space;
signaling a receiver using the signaling device to indicate the first location is a position of interest;
obtaining a first scan of the patient space using the LiDAR scanner;
determining a first virtual location of the signaling device within the patient space based on the difference between the first scan and the baseline scan; and
determining a first position of interest based on the first virtual location of the signaling device.

2. The method of claim 1, wherein the receiver is an RF receiver, and signaling the receiver is performed using an RF transmitter.

3. The method of claim 1, wherein the receiver is a microphone, and signaling the receiver is performed using a sound.

4-7. (canceled)

8. The method of claim 1, wherein the first position of interest is centered on the first location.

9. The method of claim 1, wherein the first position of interest is offset from the first location.

10. (canceled)

11. The method of claim 1, wherein the first position of interest is flagged as a keep out zone.

12-13. (canceled)

14. The method of claim 1, further comprising:
moving the signaling device at a second location within the patient space;
signaling the receiver to indicate the second location is a position of interest;
obtaining a second scan of the patient space using the LiDAR scanner;
identifying a second virtual location of the signaling device within the patient space based on a difference between the second scan and the baseline scan; and
determining a second position of interest based on the second virtual location of the signaling device.

15. The method of claim 14, further comprising merging the first position of interest and the second position of interest.

16. The method of claim 1, further comprising tracking a patient location relative to the first position of interest within the patient space.

17. The method of claim 16, further comprising providing an alert if the patient location is determined to be within the first position of interest.

18. (canceled)

19. A system for establishing virtual positions of interest in a patient space, comprising:
a processor;
a LiDAR scanner in electronic communication with the processor and configured to provide scans of the patient space;
a receiver in electronic communication with the processor;
wherein the processor is configured to:
obtain a baseline scan of the patient space from the LiDAR scanner;
obtain a first scan of the patient space when a trigger signal from a signaling device is received at the receiver;
determine a first virtual location of the signaling device within the patient space based on the difference between the first scan and the baseline scan; and
determine a first position of interest based on the first virtual location of the signaling device.

20. The system of claim 19, wherein the receiver is an RF receiver, and signaling the receiver is performed using an RF transmitter.

21. The system of claim 19, wherein the receiver is a microphone.

22. The system of claim 19, further comprising a signaling device operable to provide a signal to the receiver.

23-25. (canceled)

26. The system of claim 19, wherein the first position of interest is centered on the first location.

27. The system of claim 19, wherein the first position of interest is offset from the first location.

28. (canceled)

29. The system of claim 19, wherein the first position of interest is flagged as a keep out zone.

30-31. (canceled)

32. The system of claim 19, wherein the processor is further configured to:
obtain a second scan of the patient space when a second signal is received at the receiver;
determine a second virtual location of the signaling device within the patient space based on the difference between the first scan and the baseline scan; and
determine a second position of interest based on the second virtual location of the signaling device.

33. The system of claim 32, wherein the processor is further configured to merge the first position of interest and the second position of interest.

34. The system of claim 19, wherein the processor is further configured to:
track a patient location relative to the first position of interest within the patient space;
provide an alert if the patient location is determined to be within the first position of interest.

35-36. (canceled).
